(19) United States
(12) Patent Application Publication
(10) Pub. No.: US 2018/0133135 A1
(43) Pub. Date: May 17, 2018

(54) HAIR CLEANSING CONDITIONER

(71) Applicant: Henkel AG & Co. KGaA, Duesseldorf (DE)

(72) Inventors: Soeren Scheele, Hamburg (DE); Manuela Mette, Kleinfeld (DE); Thomas Schroeder, Hamburg (DE); Erik Schulze zur Wiesche, Hamburg (DE)

(73) Assignee: Henkel AG & Co. KGaA, Duesseldorf (DE)

(21) Appl. No.: 15/811,707

(22) Filed: Nov. 14, 2017

(30) Foreign Application Priority Data
Nov. 17, 2016 (DE) 10 2016 222 636.3

(51) Int. Cl. A61K 8/41; A61K 8/73; A61K 8/891; A61K 8/898; A61K 8/92; A61K 8/34; A61Q 5/02; A61Q 5/12

(52) U.S. Cl. CPC A61K 8/416; A61K 8/732; A61K 8/891; A61Q 5/12; A61K 8/922; A61K 8/342; A61Q 5/02; A61K 8/898

(57) ABSTRACT

Hair cleansing conditioners which—relative to the weight of the total compositions—contain
a) from about 0.01 to about 10 wt. % of at least one polyether-modified polysiloxane,
b) from about 0.01 to about 10 wt. % of at least one amino-functional silicone,
c) from about 0.01 to about 15 wt. % of at least one hydrophobically-modified starch compound, and
a) from about 0.10 to about 30 wt. % of at least one amphoteric tenside, are exemplified by an excellent cleansing and conditioning effect.

More particularly, an optimal balance between a high conditioning effect and a feeling of clean hair is achieved.

### CROSS-REFERENCE TO RELATED APPLICATION

This application claims priority to German Patent Application No. 10 2016 222 636.3, filed Nov. 17, 2016, which is incorporated herein by reference in its entirety.

### TECHNICAL FIELD

The present disclosure relates to the cosmetics sector, more particularly hair cleansing conditioners containing a polyether-modified polysiloxane, an amino/functional silicone, a hydrophobically-modified starch compound, as well as an amphoteric tenside. The present disclosure further relates to the use of the aforementioned active agent combination for achieving an optimal balance between a high conditioning effect and a clean hair feeling, as well as for improving the skin tolerance of cosmetic cleansing compositions.

### BACKGROUND

Conventional hair cleansers usually comprise anionic tensides, i.e., surfactants. These are desirable and essential to removing sebum and other contaminants from the hair surface and the scalp. However, they also remove lipids and proteins from the hair and/or scalp during the cleansing process, thereby necessitating subsequent treatment with care products in order to regenerate the hair. In addition to daily cleansing, the structure of the hair can also be damaged by environmental influences (such as intense solar irradiation), mechanical stresses (such as intense combing under the heat of a hair dryer), physical or chemical treatments (such as coloring, curling or straightening the hair). This often results in split ends, hair breakage and/or loss of hair gloss. Hair damaged in this way is difficult to disentangle and style. Hence the need for a particularly mild hair cleanser, which removes the smallest amount of grease or no grease at all from the hair during the cleansing process and nourishes it at the same time.

Patent Application U.S. Pat. No. 6,723,309 proposes a new kind of nourishing hair cleanser—referred to as a hair cleansing conditioner. This is based on a complex mixture of conditioning agents, coolants, emulsifiers, moisturizers, pH stabilizers, preservatives, thickening agents, plant extracts and vitamins. The hair cleansing conditioner cleanses the hair, lends it more shine and body and makes it easier to style. The disadvantage of the hair cleansing conditioner, however, is its weak foaming power and its sometimes poor ability to be distributed and/or rinsed off the hair. Moreover, treatment with silicone-based cleansers—particularly when regularly applied to thin hair—can have an undesirable build-up effect.

The present disclosure therefore addresses the problem of providing hair cleansing conditioners that do not have the aforementioned disadvantages. More particularly, of providing hair cleansing conditioners with effective foaming power, which ideally contain no anionic (sulphate) tensides, yet nonetheless guarantee thorough and particularly mild cleansing. A further aim was to find suitable silicone-based active agents for the hair cleansing conditioner, which do not have any build-up effect, even after regular application to the hair.

### BRIEF SUMMARY

A hair cleansing conditioner is provided in an exemplary embodiment. The hair cleansing conditioner includes from about 0.01 to about 10 wt. % of at least one polyether-modified polysiloxane, relative to the weight of the entire composition. The hair cleansing conditioner includes from about 0.01 to about 10 wt. % of at least one amino-functional silicone, relative to the weight of the entire composition. The hair cleansing conditioner also includes from about 0.01 to about 15 wt. % of at least one hydrophobically-modified starch compound, relative to the weight of the entire composition. Further, the hair cleansing conditioner includes from about 0.10 to about 30 wt. % of at least one amphoteric tenside, relative to the weight of the entire composition.

In another embodiment, a cosmetic use of an active ingredient combination is provided in hair cleansing conditioners for achieving an optimal balance between a high conditioning effect and a feeling of clean hair or for improving the skin tolerance of cosmetic hair cleansing compositions. The exemplary active ingredient combination includes from about 0.01 to about 10 wt. % of at least one polyether-modified polysiloxane, from about 0.01 to about 10 wt. % of at least one amino-functional silicone, from about 0.01 to about 15 wt. % of at least one hydrophobically-modified starch compound, and from about 0.10 to about 30 wt. % of at least one amphoteric tenside.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

Surprisingly, it has now emerged that the aforementioned problems are ideally solved by hair cleansing conditioners which, in addition to two special silicone compounds, contain at least one hydrophobically-modified starch compound and an amphoteric tenside.

Such hair cleansing conditioners form mild cleansing products with very good foaming and conditioning properties, which lend the hair gloss and resilience even with repeated application, without the build-up effect. The hair cleansing conditioners achieve an optimal balance between a high conditioning effect and a clean hair feeling, and are well tolerated by the skin.

A first subject matter of the present application is a hair cleansing conditioner which—relative to the weight of the total composition—contains
a) from about 0.01 to about 10 wt. % of at least one polyether-modified polysiloxane,
b) from about 0.01 to about 10 wt. % of at least one amino-functional silicone,
c) from about 0.01 to about 15 wt. % of at least one hydrophobically-modified starch compound and
d) from about 0.10 to about 30 wt. % of at least one amphoteric tenside.

The hair cleansing conditioners as contemplated herein contain the active agents a) to d), such as in a suitable cosmetic carrier. As contemplated herein, an exemplary cosmetic carrier is a hydrous carrier, which (relative to the total weight of the hair cleansing conditioner) contains at least about 55 wt. %, such as at least about 60 wt. %, for example at least about 65 wt. %, such as at least about 70 wt. % of water. Furthermore, the cosmetic carrier can contain from about 0.01 to about 30 wt. %, such as from about 0.05 to about 20 wt. %, for example from about 0.1 to about 10 wt. % of at least one alcohol. Suitable alcohols include ethanol, ethyldiglycol, 1-propanol, 2-propanol, isopropanol, 1,2-propylenglycol, 1,3-propylenglycol, 1-butanol, 2-butanol, 1,2-butandiol, 1,3-butandiol, 1,4-butandiol, 1-pentanol, 2-pentanol, 1,2-pentandiol, 1,5-pentandiol, 1, hexanol, 2-hexanol, 1,2-hexandiol, 1,6-hexandiol, glycerine, polyethylenglycol, sorbitol, sorbitan, benzyl alcohol, phenoxyethanol or mixtures of said alcohols. In certain embodiments, the cosmetic carrier contains hydrous alcohols, such as ethanol, isopropanol, 1,2-propylenglycol, glycerine, sorbitol, benzyl alcohol and/or phenoxyethanol, as well as combinations of said alcohols. For example, in certain embodiments, the cosmetic carrier contains glycerine, isopropanol and/or 1,2-propylenglycol.

The hair cleansing conditioners as contemplated herein contain as the constituent a) at least one polyether-modified polysiloxane in a percentage by weight of from about 0.01 to about 10 wt. % of the total weight of the hair cleansing conditioner. The at least one silicone a) may be used in a percentage by weight of from about 0.025 to about 8.00 wt. % (such as from about 0.05 to about 6.00 wt. %, for example from about 0.075 to about 4.00 wt. %, such as from about 0.10 to about 2.00 wt. %) of the total weight of the hair cleansing conditioner.

Suitable polyether-modified polysiloxanes a) may be compounds according to the following formula (I), in which

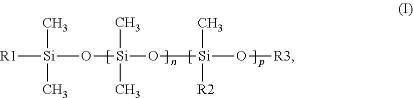

Radicals R1 and R3 independently constitute a hydrogen atom, an alkyl group with from about 1 to about 30 C-atoms, an alkoxy group with from about 1 to about 30 C-atoms or a possibly substituted phenyl group,
Radical R2 constitutes the group —C_{c}H_{2c}—O—(C₂H₄O—)ₐ(C₃H₆O—)_{b}R5,
Radical R5 constitutes a hydrogen atom or a linear or branched alkyl group with 1 to 16 C-atoms,
n is a number from about 0 to about 500,
p is a number from about 1 to about 50,
a is a number from about 0 to about 50,
b is a number from about 0 to about 50,
a+b are at least 1, and
c is a number from about 1 to about 4.

In certain embodiments, polyether-modified polysiloxanes of the general structural formula (I) are:

TABLE 1

| | R1, R3 | R2 | R5 | n | p | a | b | c |
|---|---|---|---|---|---|---|---|---|
| 1 | H, methyl | —C_{c}H_{2c}—O—(C₂H₄O)ₐ—(C₃H₆O)_{b}—R₅ | H, methyl | 1-500 | 5-50 | 15-25 | 15-25 | 3 |
| 2 | H, methyl | —C_{c}H_{2c}—O—(C₂H₄O)ₐ—(C₃H₆O)_{b}—R₅ | H, methyl | 1-500 | 5-50 | 5-20 | 0.1 | 3 |
| 3 | H, methyl | —C_{c}H_{2c}—O—(C₂H₄O)ₐ—(C₃H₆O)_{b}—R₅ | H, methyl | 0.1 | 1-3 | 5-10 | 0.1 | 2 |
| 4 | H, methyl | —C_{c}H_{2c}—O—(C₂H₄O)ₐ—(C₃H₆O)_{b}—R₅ | H, methyl | 1-500 | 5-50 | 20-30 | 20-30 | 3 |
| 5 | H, methyl | —C_{c}H_{2c}—O—(C₂H₄O)ₐ—(C₃H₆O)_{b}—R₅ | H, methyl | 1-500 | 5-50 | 10-25 | 10-25 | 3 |
| 6 | H, methyl | —C_{c}H_{2c}—O—(C₂H₄O)ₐ—(C₃H₆O)_{b}—R₅ | H, methyl | 1-500 | 2-50 | 10-30 | 2-10 | 3 |
| 7 | H, methyl | —C_{c}H_{2c}—O—(C₂H₄O)ₐ—(C₃H₆O)_{b}—R₅ | H, methyl | 1-500 | 2-50 | 10-20 | 1-10 | 3 |

In certain embodiments, polyether-modified polysiloxanes of the general structural formula (I) are:

TABLE 2

| | R1, R3 | R2 | R5 | n | p | a | b | c |
|---|---|---|---|---|---|---|---|---|
| 1 | Methyl | —C_{c}H_{2c}—O—(C₂H₄O)ₐ—(C₃H₆O)_{b}—R₅ | H, methyl | 5-500 | 7-50 | 15-20 | 15-20 | 3 |
| 2 | Methyl | —C_{c}H_{2c}—O—(C₂H₄O)ₐ—(C₃H₆O)_{b}—R₅ | H, methyl | 5-500 | 7-50 | 7-15 | 0 | 3 |
| 3 | Methyl | —C_{c}H_{2c}—O—(C₂H₄O)ₐ—(C₃H₆O)_{b}—R₅ | H, methyl | 0.1 | 1 | 6-8 | 0 | 2 |
| 4 | Methyl | —C_{c}H_{2c}—O—(C₂H₄O)ₐ—(C₃H₆O)_{b}—R₅ | H, methyl | 5-500 | 7-50 | 20-25 | 20-25 | 3 |
| 5 | Methyl | —C_{c}H_{2c}—O—(C₂H₄O)ₐ—(C₃H₆O)_{b}—R₅ | H, methyl | 5-500 | 7-50 | 15-20 | 15-20 | 3 |
| 6 | Methyl | —C_{c}H_{2c}—O—(C₂H₄O)ₐ—(C₃H₆O)_{b}—R₅ | H, methyl | 5-500 | 2-50 | 15-25 | 2-8 | 3 |
| 7 | Methyl | —C_{c}H_{2c}—O—(C₂H₄O)ₐ—(C₃H₆O)_{b}—R₅ | H, methyl | 5-500 | 2-50 | 10-15 | 1-5 | 3 |

In certain embodiments, polyether-modified polysiloxanes of the general structural formula (I) are:

TABLE 3

| | R1, R3 | R2 | R5 | n | p | a | b | c |
|---|---|---|---|---|---|---|---|---|
| 1 | Methyl | —C_{c}H_{2c}—O—(C₂H₄O)ₐ—(C₃H₆O)_{b}—R₅ | Methyl | 10-500 | 10-50 | 18 | 18 | 3 |
| 2 | Methyl | —C_{c}H_{2c}—O—(C₂H₄O)ₐ—(C₃H₆O)_{b}—R₅ | Methyl | 10-500 | 10-50 | 12 | 0 | 3 |
| 3 | Methyl | —C_{c}H_{2c}—O—(C₂H₄O)ₐ—(C₃H₆O)_{b}—R₅ | Methyl | 10-500 | 10-50 | 14 | 0 | 3 |
| 4 | Methyl | —C_{c}H_{2c}—O—(C₂H₄O)ₐ—(C₃H₆O)_{b}—R₅ | Methyl | 0 | 1 | 7 | 0 | 2 |
| 5 | Methyl | —C_{c}H_{2c}—O—(C₂H₄O)ₐ—(C₃H₆O)_{b}—R₅ | Methyl | 10-500 | 10-50 | 22 | 24 | 3 |
| 6 | Methyl | —C_{c}H_{2c}—O—(C₂H₄O)ₐ—(C₃H₆O)_{b}—R₅ | Methyl | 10-500 | 10-50 | 17 | 18 | 3 |
| 7 | Methyl | —C_{c}H_{2c}—O—(C₂H₄O)ₐ—(C₃H₆O)_{b}—R₅ | Methyl | 10-500 | 5-50 | 20 | 6 | 3 |
| 8 | Methyl | —C_{c}H_{2c}—O—(C₂H₄O)ₐ—(C₃H₆O)_{b}—R₅ | Methyl | 10-500 | 5-50 | 14 | 4 | 3 |

Polyether-modified polysiloxane compounds a), which are listed in the aforementioned Tables 1-3, are commercially available under the following trade names, for example
1: Dow Corning 190 (INCI: PEG/PPG-18/18 Dimethicone),
2: Dow Corning 193 (INCI: PEG-12 Dimethicone),
3: ABIL® B 8843 (INCI: PEG-14 Dimethicone),
4: Silwet L-77,
5: Mirasil DCMO (INCI: Cyclomethicone, PEG/PPG-22/24 Dimethicone),
6: Dow Corning Q2-5220 (INCI: PEG/PPG-17/18 Dimethicone),
7: ABIL® B 88184 (INCI: PEG/PPG-20/6 Dimethicone), and
8: ABIL® B 8851 (INCI: PEG/PPG-14/4 Dimethicone).

Certain exemplary hair cleansing conditioners are exemplified in that they contain at least one polyether-modified polysiloxane compound a) according to the formula (I), which fulfill the conditions in Line 2 (Tables 1-3) (for example Dow Corning 193 or Xiameter® OFX 0193 (INCI: PEG-12 Dimethicone)).

Likewise certain exemplary hair cleansing conditioners are exemplified in that they contain at least one polyether-modified polysiloxane compound a) according to the formula (I), which fulfill the conditions in Line 2 in Tables 1 and 2, and also in Table 3 (for example ABIL® B 8843 (INCI: PEG-14 Dimethicone)).

The compounds available under the INCI trade names PEG-12 Dimethicone and PEG-14 Dimethicone can be used in the hair cleansing conditioners as contemplated herein, both individually and also as mixtures, in the aforementioned quantities.

The hair cleansing conditioners as contemplated herein contain as the constituent b) at least one amino-functional silicone in a percentage by weight of from about 0.01 to about 10 wt. % of the total weight of the hair cleansing conditioner. In an exemplary embodiment, the at least one silicone b) is used in a percentage by weight of from about 0.025 to about 8.50 wt. % (such as from about 0.05 to about 7.00 wt. %, for example from about 0.075 to about 5.00 wt. %, such as from about 0.10 to about 2.50 wt. %) of the total weight of the hair cleansing conditioner.

In certain embodiments, suitable amino-functional silicones b) are compounds according to the following formula (II),

Si(CH₃)₃—[O—Si(CH₃)₂]ₓ—[O—Si(CH₃)(R—NH—CH₂—CH₂—NH₂)]_{y}—O—Si(CH₃)₃ (II),

in which
x+y is a number from about 50 to about 500,
the amino-functionality is from about 0.03 to about 8 wt. %, and
R is an alkylene group with from about 2 to about 5 carbon atoms.

In certain embodiments, suitable amino-functional silicones b) according to the Formula (II) are those wherein
x+y is a number from about 75 to about 400, such as a number from about 100 to about 300,
the amino functionality is from about 0.50 to about 4 wt. %, such as from about 1.00 to about 2 wt. % (measured and calculated as described below), and
Radical R denotes an ethylene, propylene, isopropylene, butylene, isobutylene or pentylene grouping.

The wt. % of the amino functionality can be measured by titrating a sample of the amino-functionalized silicone against alcoholic hydrochloric acid through to the bromocresol green end point, wherein the wt. % of amine is calculated by applying a molecular weight of 45 (according to CH₃—CH₂—NH₂).

Certain exemplary hair cleansing conditioners are exemplified in that they contain at least one amino-functional silicone compound b) according to Formula (II) in the aforementioned quantities, wherein
x+y is a number from about 100 to about 300,
the (measured and calculated) amino functionality is from about 1.00 to about 2 wt. %, and
Radical R denotes an ethylene, propylene, isopropylene, butylene, isobutylene or pentylene grouping.
   An example of such an amino-functional silicone compound b) is the compound commercially available under the trade name Dow Corning® 2-8566 Amino Fluid (INCI: Amodimethicone).

The hair cleansing conditioners as contemplated herein contain as the constituent c) at least one hydrophobically-modified starch compound in a percentage by weight of from about 0.01 to about 15 wt. % of the total weight of the hair cleansing conditioner. In an exemplary embodiment, the at least one hydrophobically-modified starch compound c) is used in a percentage by weight of from about 0.10 to about 12.50 wt. % (such as from about 0.25 to about 10.00 wt. %, for example from about 0.50 to about 5.00 wt. %, such as from about 0.50 to about 5.00 wt. %) of the total weight of the hair cleansing conditioner.

Suitable starch compounds according to exemplary embodiments are plant-based and may be produced from corn, wheat, rice, tapioca, potatoes and/or sago. As contemplated herein, “hydrophobic modification” means chemical cross-linking, for example by employing ionic cross-linking with calcium, aluminum and/or phosphates—for example, in certain embodiments with phosphates—and/or chemical modification by introducing hydrophic groups. Exemplary hydrophobic groups are non-ionic radicals such as hydroxyalkyl groups, wherein exemplary “hydroxyalkyl groups” are hydroxyethyl, hydroxypropyl and/or hydroxybutyl groups. As contemplated herein, exemplary starch compounds are cross-linked and carry hydrophobic, non-ionic substitutes, such as hydroxypropyl groups. Exemplary starch compounds are the hydrophobically-modified starch compounds known under the INCI trade names of hydroxyethyl starch phosphate and hydroxypropyl starch phosphate. An exemplary starch compound is hydroxypropyl starch phosphate.

Exemplary hydrophobically-modified starch compounds c) are solid and may have medium particle sizes in the range of from about 1 μm to about 100 μm, such as from about 5 μm to about 50 μm, for example from about 10 μm to about 40 μm.

An example of a hydrophobically-modified starch compound c) are the compounds Structure® XL (AkzoNobel), Structure® ZEA (National Starch) and/oder Farinex® VA 70 (Avebe U.A.) known under the INCI trade name of hydroxypropyl starch phosphate and available from several suppliers.

In an exemplary embodiment, a starch phosphate substituted with hydroxyalkyl groups, such as hydroxypropyl starch phosphate, is used as the hydrophobically-modified starch compound c).

Use of a compound known under the INCI trade name hydroxypropyl starch phosphate in the aforementioned quantities in the hair cleansing conditioners is contemplated herein.

The hair cleansing conditioners as contemplated herein contain as the constituent d) at least one amphoteric tenside in a percentage by weight of from about 0.10 to about 30 wt. % of the total weight of the hair cleansing conditioner. The at least one amphoteric tenside d) may be used in a percentage by weight of from about 0.50 to about 25.00 wt. %, such as from about 0.75 to about 20.00 wt. %, for example from about 1.00 to about 17.50 wt. %, such as from about 1.50 to about 15.00 wt. %, of the total weight of the hair cleansing conditioner.

A content of active cleansing—such as mild—amphoteric tenside d) may be utilized to achieve adequate foaming quantities and qualities. As contemplated herein, “amphoteric” tensides are zwitterionic tensides.

Suitable amphoteric and/or zwitterionic tensides d) according to the present disclosure can be selected from one or several compounds of the following formulas (i) to (vii), wherein Radical R denotes, in each case, a straight-chained or branched, saturated or mono- or poly unsaturated alkyl- or alkenyl radical with from about 7 to about 23 carbon atoms (Formulas (i) and (ii)) or a straight-chained or branched, saturated or mono- or poly unsaturated alkyl- or alkenyl radical with from about 8 to about 24 carbon atoms (Formulas (iii) to (vii):

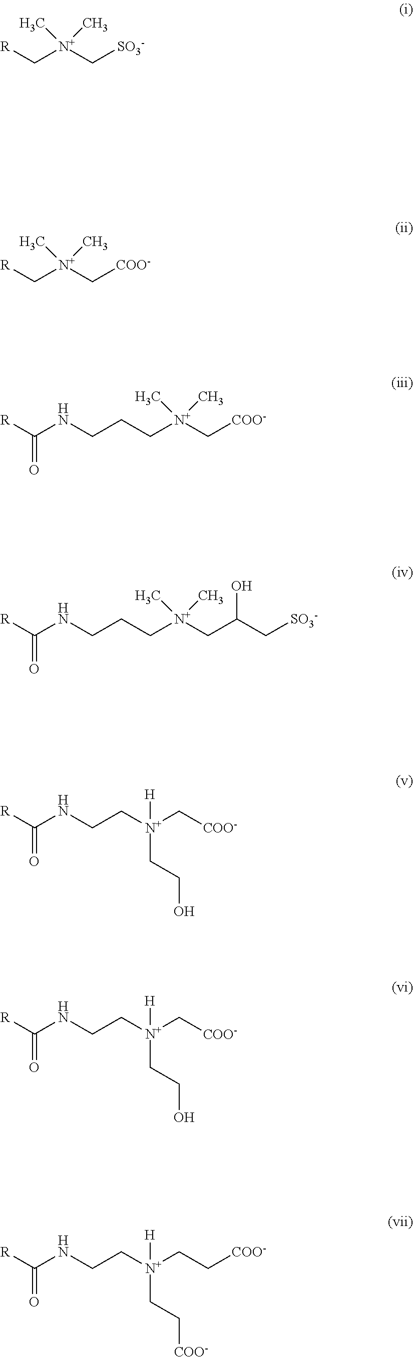

Exemplary amphoteric and/or zwitterionic tensides of one of the above Formulas (i) to (vii) contain primarily as Radical R a straight-chained or branched, saturated, mono- or poly unsaturated alkyl radical with from about 8 to about 20, such as from about 8 to about 18, for example from about 8 to about 16 C-atoms. In exemplary amphoteric and/or zwitterionic tensides, Radical R is derived from coconut oil. Exemplary amphoteric/zwitterionic tensides are known under the INCI trade names of sodium cocoamphoacetate, disodium cocoamphodiacetate, sodium lauroamphoacetate, sodium lauroamphodiacetate, sodium cocoamphopropionate, disodium cocoamphodipropionate, coco betaine, lauryl betaine, cocamidopropylbetain and/or cauramidopropylbetain and commercially available from several suppliers. Tensides with the INCI trade names of cocamidopropylbetain and cocoampho(di)acetate can be used in the aforementioned quantities in the hair cleansing conditioners as contemplated herein either individually or as a mixture.

To further optimize the foam quality, as well as the hair-nourishing properties, the hair cleansing conditioners as contemplated herein may additionally contain at least one cationic tenside e), such as a cationic tenside of the quarternary ammonium compound type.

Exemplary quarternary ammonium compounds are ammoniumhalogenides, for example chlorides and bromides, such as alkyltrimethylammoniumchlorides, dialkyldimethylammoniumchlorides and trialkylmethyl-ammoniumchlorides, e.g. lauryltrimethylammoniumchloride, cetyltrimethylammoniumchloride, cetyltrimethylammoniumbromide, cetyltrimethyl-ammoniummethosulphate, dicetyldimethylammoniumchloride, tricetylmethyl-ammoniumchloride, stearyltrimethyl-ammoniumchloride, distearyldimethylammoniumchloride, lauryl-dimethylbenzylammoniumchloride, behenyltrimethylammoniumchloride, behenyltri-methylammoniumbromide and/or behenyltrimethylammoniummethosulphate, as well as the imidazolium compounds known under the INCI trade names of Quaternium-27 and Quaternium-83. Exemplary long alkyl chains of the aforementioned tensides have from about 10 to about 22, such as from about 12 to about 22, for example from about 14 to about 22, such as from about 16 to about 22 carbon atoms. Suitable compounds are known under the INCI trade names of behentrimonium chloride and/or cetrimoniumchloride.

In an exemplary embodiment of the present disclosure, the hair cleansing conditioners contain at least one cationic tenside e) in a percentage by weight of from about 0.10 to about 10.00 wt. %, such as from about 0.25 to about 9.00 wt. %, for example from about 0.40 to about 7.50 wt. %, such as from about 0.50 to about 5.00 wt. %, of the total weight of the hair cleansing conditioners. In certain embodiments, the hair cleansing conditioners contain as a cationic tenside e) at least one of the compounds known under the INCI trade names of cetrimoniumchloride and/or behentrimoniumchloride in the aforementioned quantities.

It has been determined that a very good balance between a high foam quantity, pleasant feel of the foam and hair nourishment can be achieved if the hair cleansing conditioners as contemplated herein contain a combination of
d) cocoampho(di)acetate and/or cocamidopropylbetain with
e) behentrimoniumchloride and/or cetrimoniumchloride in the aforementioned quantities.
   In such embodiments, it is not necessary for further tensides, more particularly anionic tensides, to be added to the hair cleansing conditioners to increase the foaming power.

In a further embodiment, the hair cleansing conditioners as contemplated herein are therefore essentially free of anionic tensides.

Within this embodiment, the hair cleansing conditioners as contemplated herein may be essentially free of anionic sulphate tensides.

“Essentially free from” means a content of anionic (sulphate) tensides and/or silicones of maximum 0.50 wt. %, for example of maximum 0.30 wt. %, such as of maximum 0.10 wt. %, for example 0 wt. %, wherein the quantity values refer to the total weight of the hair cleansing conditioners as contemplated herein. The total maximum quantities apply to anionic (sulphate) tensides that can be added to the hair cleansing conditioners as contemplated herein either in their free form and/or as hydrous solutions and/or to silicones that can be added in their free form and/or as emulsions or dispersions, and not to any quantities that can be contained in various commercial products in subordinate quantities as a minor component.

For certain embodiments, further hair-conditioning active ingredients f) may be added to the hair cleansing conditioners as contemplated herein in order to increase and/or optimize the nourishing properties. Exemplary nourishing active ingredients f) may be capable of being easily incorporated into the hair cleansing conditioners as contemplated herein and may not negatively influence the stability and/or foaming properties thereof. In such embodiments, the combination of the further nourishing agents f) and the active ingredients a) through d) are selected to not cause an over-conditioning of the hair, which can lead to an undesirable hardening of the hair and an undesirable build-up effect with regular application.

The following hair-conditioning active ingredients f) have proven to be particularly advantageous
vitamins (fi) and/or
oil, grease and/or wax components (fii).

Suitable oil, wax and/or fatty components (fii) can be selected from mineral and/or natural oil components and/or from fatty substances. Triglycerides and triglyceride compounds are normally used as natural (plant) oils. Exemplary natural oils are coconut oil, (sweet) almond oil, walnut oil, peach kernel oil, apricot kernel oil, avocado oil, tee tree oil, soja oil, sesame oil, sunflower oil, tsubaki oil, evening primrose oil, rice bran oil, palm kernel oil, mango kernel oil, meadowfoam seed oil, thistle oil, macadamia nut oil, grape seed oil, amaranth seed oil, argan oil, bamboo oil, olive oil, wheatgerm oil, pumpkin seed oil, mallow oil, hazelnut oil, safflower oil, canola oil, sasanqua oil, jojoba oil, rambutan oil, cocoa butter and shea butter. Mineral oils, paraffin and iso-paraffin oils, as well as synthetic hydrocarbons may be used as mineral oils. An example of a usable hydrocarbon is the commercially available 1,3-Di-(2-ethylhexyl)-cyclohexan (Cetiol® S). A dialkylether can also be used as an oil component. Usable dialkylethers are particularly di-n-alkylether with a total of between 12 and 36 C-atoms, more particularly 12 to 24 C-atoms, such as for example di-n-octylether, di-n-decylether, di-n-nonylether, di-n-undecylether, di-n-dodecylether, n-hexyl-n-octylether, n-octyl-n-decylether, n-decyl-n-undecylether, n-undecyl-n-dodecylether and n-hexyl-n-undecylether, as well as di-tert.-butylether, di-iso-pentylether, di-3-ethyldecylether, tert.-butyl-n-octylether, iso-pentyl-n-octylether and 2-methylpentyl-n-octylether. An exemplary dialkylether is the commercially available di-n-octylether under the trade name Cetiol® OE. Fatty substances are fatty acids, fatty alcohols, as well as natural and synthetic waxes, which can exist both in solid form and liquid form on hydrous dispersions. Linear and/or branched, saturated and/or unsaturated fatty acids with from about 6-about 30 carbon atoms can be used as fatty acids. Fatty acids with from about 10-about 22 carbon atoms are suitable. These include the isostearic acids, such as the commercially available Emersol® 871 and Emersol® 875, and isopalmitic acids, such as the commercially available Edenor® IP 95, as well as all other fatty acids sold under the trade name Edenor® (Cognis). Other typical examples of such fatty acids are caproic acid, caprylic acid, 2-ethyl hexane acid, capric acid, lauric acid, isotridecaric acid, myristiric acid, palmitic acid, palmitoleic acid, stearic acid, isostearic acid, oleic acid, elaidic acid, petroselic acid, linoleic acid, linolenic acid, elaeostearic acid, arachine acid, gadoleic acid, behen acid and eruca acid, as well as the technical mixtures thereof. Exemplary embodiments utilize the fatty acid fractions that can be obtained from coconut oil or palm oil, for example stearic acid. Saturated, mono- or poly-unsaturated, branched or unbranched fatty alcohols with C₆-C₃₀—, such as C₁₀-C₂₂, for example C₁₂-C₂₂, carbon atoms, can be used as fatty alcohols. Decanol, octanol, octenol, dodecenol, decenol, octadienol, dodecadienol, decadienol, oleylalcohol, erucaalcohol, ricinolalcohol, stearylalcohol, isostearylalcohol, cetylalcohol, laurylalcohol, myristylalcohol, arachidylalcohol, caprylalcohol, caprinalcohol, linoleylalcohol, linolenylalcohol and behenylalcohol, as well as the guerbetalcohols thereof, can be used. This list contains purely examples and is not exhaustive. The fatty alcohols originate, however, from natural fatty acids, wherein the recovery from the esters of fatty acids by employing reduction can normally be assumed. As contemplated herein, fatty alcohol fractions produced by reducing naturally-occurring triglycerides such as beef tallow, palm oil, peanut oil, rapeseed oil, cotton seed oil, soya oil, sunflower oil and linseed oil, or the fatty acid esters produced from the trans-esterification products with corresponding alcohols can also be used. These substances therefore constitute a mixture of different fatty alcohols. Such substances include those commercially available under the trade names of Stenol®, e.g. Stenol® 1618 or Lanette®, e.g. Lanette® O or Lorol®, e.g. Lorol® C8, Lorol® C14, Lorol® C18, Lorol® C8-18, HD-Ocenol®, Crodacol®, z.B. Crodacol® CS, Novol®, Eutanol® G, Guerbitol® 16, Guerbitol® 18, Guerbitol® 20, Isofol® 12, Isofol® 16, Isofol® 24, Isofol® 36, Isocarb® 12, Isocarb® 16 oder Isocarb® 24. As contemplated herein, wool wax alcohols, such as those commercially available under the trade names of Corona®, White Swan®, Coronet® or Fluilan®, can of course also be used. Solid paraffins or iso-paraffins, carnauba waxes, beeswaxes, candelilla waxes, ozokerites, ceresine, spermaceti, sunflower wax, fruit wax, such as apple wax or citrus wax, micro-waxes from PE- or PP can also be used as natural or synthetic waxes. Such waxes are available from Kahl & Co., Trittau, for example. Other solids include
Ester oils. Ester oils include the esters from C6-C30 fatty acids with C2-C30—fatty alcohols. The mono esters of fatty acids with alcohols with from about 2 to about 24 C-atoms are suitable. Examples of used fatty acid fractions in the esters are caproic acid, caprylic acid, 2-ethyl hexane acid, capric acid, lauric acid, isotridecaric acid, myristiric acid, palmitic acid, palmitoleic acid, stearic acid, isostearic acid, oleic acid, elaidic acid, petroselic acid, linolic acid, linolenic acid, elaeostearic acid, arachine acid, gadoleic acid, behen acid and eruca acid, as well as the technical mixtures thereof. Examples of the fatty alcohol fractions in the ester oils are isopropylalcohol, capronalcohol, aprylalcohol, 2-ethylhexylalcohol, caprinalcohol, laurylalcohol, isotridecylalcohol, myristylalcohol, cetylalkohol, palmoleylalcohol, stearylalcohol, isostearylalcohol, oleylalcohol, elaidylalcohol, petroselinylalcohol, linolylalcohol, linolenylalcohol, elaeostearylalcohol, arachylalcohol, gadoleylalcohol, behenylalcohol, erucylalcohol and brassidylalcohol, as well as the technical mixtures thereof. Isopropylmyristat (Rilanit® IPM), Isononan acid-C16-18-alkylester (Cetiol® SN), 2-ethylhexylpalmitat (Cegesoft® 24), stearic acid-2-ethylhexylester (Cetiol® 868), eetyloleate, glycerintricaprylate, coconut fatty alcohol-caprinate/-caprylat (Cetiol® LC), n-butylstearate, oleylerucate (Cetiol J 600), isopropylpalmitate (Rilanit® IPP), Oleyl Oleate (Cetiol®), lauric acid hexylester (Cetiol® A), Di-n-butyladipat (Cetiol® B), myristylmyristate (Cetiol® MM), cetearyl isononanoate (Cetiol® SN), oleic acid decylester (Cetiol® V),
Dicarbonic acid esters such as di-n-butyladipate, ei-(2-ethylhexyl)-adipate, di-(2-ethylhexyl)-succinate and Di-isotridecylacelaate, as well as diolesters such as ethylenglykol-dioleate, ethylenglykol-di-isotridecanoate, propylenglykol-di(2-ethylhexanoate), propylenglykol-di-isostearate, propylenglykol-di-pelargonate, butandiol-di-isostearate, neopentylglykoldicaprylate,
symmetrical, asymmetrical or cyclical esters of carbonic acids with fatty alcohols,
glycerincarbonate or dicaprylylcarbonate (Cetiol® CC),
ethoxylated or non-ethoxylated mono,- di- and tri-fatty acid esters of saturated and/or unsaturated linear and/or branched fatty acids with glycerine, such as Monomuls® 90-O18, Monomuls® 90-L12, Cetiol® HE or Cutina® MD.

Exemplary oil, wax and/or fatty components (fii) according to this present disclosure are selected from fatty alcohols and/or plant oils due to properties thereof, which nourish and which have no negative effect on the stability of the compositions as contemplated herein. Examples of suitable constituents (fii) are cetyl alcohol, stearyl alcohol, cetearyl alcohol and/or one or more of the aforementioned plant oils.

The proportions by weight of the oil, wax and/or fatty components (fii) of the total weight of the hair cleansing conditioners according to exemplary embodiments herein are from about 0.01 to about 10.00 wt. %, such as from about 0.25 to about 9.00 wt. %, for example from about 0.50 to about 8.00 wt. %, such as from about 0.75 to about 7.50 wt. %.

In a further exemplary embodiment, the hair cleansing conditioners as contemplated herein contain—relative to their weight—from about 0.01 to about 10.00 wt. %, such as from about 0.25 to about 9.00 wt. %, for example from about 0.50 to about 8.00 wt. % such as from about 0.75 to about 7.50 wt. % of at least one saturated or unsaturated, linear or branched C12-C24 alcohol, such as selected from the compounds known under the INCI trade names cetyl alcohol, stearyl alcohol and/or cetearyl alcohol.

Among the suitable vitamins (fi) are the following vitamins, provitamins and vitamin precursors, as well as the derivative thereof:
Vitamin A: The group of substances called Vitamin A include retinol (Vitamin A₁) and 3,4-didehydroretinol (Vitamin A₂). β-carotin is the provitamin of retinal. Vitamin A-components can include Vitamin A-acid and the esters thereof, Vitamin A-aldehyd and Vitamin A-alkohol, as well as the esters thereof, such as palmitate and acetate.
Vitamin B: The Vitamin B Group or Vitamin B complex include
Vitamin B₁ (Thiamin)
Vitamin B₂ (Riboflavin)
Vitamin B₃. The compounds nicotinic acid amide (niacin amide) are often carried under this designation.
Vitamin B₅ (pantothenic acid and panthen oil). Among this group, panthenol is used in certain embodiments. Usable derivatives of panthenol include, in particular, the esters and ethers of panthenol, pantolacton, as well as cationically derivatized panthenols. Specific examples are panthenoltriacetate, panthenolmonoethylether and the monoacetate thereof, as well as cationic panthenol derivatives.
Vitamin B₆ (pyridoxine, as well as pyridoxamine and pyridoxal).
Vitamin C (ascorbic acid): use in the form of palmitic acid ester, glucoside or phosphate in certain embodiments, and used in combination with tocopherolene in certain embodiments.
Vitamin E (tocopheroles, more particularly α-tocopherole).
Vitamin F: The term “Vitamin F” usually means essential fatty acids, more particularly linoleic acid, linolenic acid and arachidon acid.
Vitamin H: The compound (3aS,4S,6aR)-2-oxohexahydrothienol[3,4-d]-imidazol-4-valeric acid, which has however since taken on the trivial name of biotin, is designated as Vitamin H.

Vitamins, provitamins and vitamin precursors (fi) from the groups A, B, E and H are used in certain embodiments, such as nicotinic acid amide, biotin, pantolactone and/or panthenol. The proportion by weight of the vitamins(s), vitamin derivatives(e), and/or the vitamin precursor(s) (ei) of the total weight of the hair cleansing conditioners according to certain embodiments is from about 0.005 to about 1.00 wt. %, such as from about 0.01 to about 0.50 wt.-%.

In a further exemplary embodiment, the hair cleansing conditioners as contemplated herein contain—relative to their weight—additionally from about 0.001 to about 2.00 wt. %, such as from about 0.005 to about 1.00 wt. %, for example from about 0.01 to about 0.50 wt. % of at least one vitamin, one vitamin derivative and/or a vitamin precursor, for example selected from nicotinic acid amide and/or panthenol.

The hair cleansing conditioners according to exemplary embodiments here are conventional rinse-off products, such as a nourishing shampoo and/or a foaming, rinse-off nourishing conditioner. In other embodiments, the hair cleansing conditioners as contemplated herein are applied as cleansing hair foams. This guarantees a particularly good distribution and dosability of the hair cleansing conditioners.

The hair cleansing conditioners as contemplated herein therefore exist in certain embodiments as cleansing nourishing foam.

For this purpose, the hair cleansing conditioners as contemplated herein are packaged in a dispensing device, which constitutes either a compressed gas container (aerosol container) additionally filled with a propellant, or a non-aerosol container. The compressed gas container, by employing which a product is distributed through a valve by the gas pressure inside the container, is defined as an “aerosol container”. Conversely, a container under normal pressure, by employing which a product is distributed by a pumping or squeezing system via a mechanical effect, is defined as a “non-aerosol container”. Within this embodiment, the hair cleansing conditioners typically exist as an aerosol foam. Exemplary hair cleansing conditioners therefore also contain at least one propellant. The hair cleansing conditioners as contemplated herein, which exist in the form of an aerosol product, can be manufactured in a usual manner. As a rule, all constituents of the hair cleansing conditioners as contemplated herein, with the exception of the propellant, are placed in a suitable pressure-resistant container. This is then closed by employing a valve. The desired quantity of propellant is then added by employing conventional technology. In the embodiment as an aerosol foam, suitable propellants as contemplated herein are selected from N₂O, dimethylether, CO₂, air, alkanes with 3 to 5 carbon atoms, such as propane, n-butane, iso-butane, n-pentane and iso-pentane, and the mixtures thereof. According to the embodiment of an aerosol foam, the stated alkanes, mixtures of said alkanes or mixtures of said alkanes with dimethyl ether are used as the only propellant. However, the present disclosure also explicitly comprises the co-utilization of propellants of the type of fluorochloro hydrocarbons, more particularly however, the fluoro hydrocarbons. Dimethylether, propane, n-butane, iso-butane and the mixtures thereof are used in certain embodiments. Mixtures of propane and butane are used in certain embodiments as the only propellant in the propane to butane weight ratio from about 70 to about 30 to about 15 to about 85. These mixtures may be used in the hair cleansing conditioners as contemplated herein in a quantity of from about 1.00 to about 50.0 wt. %—relative to the weight of the total hair cleansing conditioners, such as quantities of from about 2.00 to about 40.0 wt. %, for example from about 3.00 to about 30.0 wt. %, such as from about 4.00 to about 20.0 wt. %. As contemplated herein, butane means n-butane, iso-butane and mixtures of n-butane and iso-butane.

In a further embodiment, the hair cleansing conditioners as contemplated herein exist as aerosol foam in an aerosol container and also contain at least one propellant. Within such an embodiment, it may be advantageous for the hair cleansing conditioners to contain the propellant—such as a mixture from propane/butane—in a percentage by weight of from about 1.00 to about 50.0 wt. %, such as from about 2.00 to about 40.0 wt. %, for example from about 3.00 to about 30.0 wt. %, such as from about 4.00 to about 20.0 wt. % of the total weight of the composition.

Other active ingredients, adjuvants and additives, which may be contained in the hair cleansing conditioners according to certain embodiments, include:
Cationically-modified protein hydrolysates, where applicable
Non-ionic tensides, particularly alkyl(oligo)glycosides, aminoxides and/or fatty acid alkanolamide,
Cationic polymers, particularly those compounds known under the INCI trade names of guar hydroxypropyltrimonium chloride, polyquaternium-10, polyquaternium-6, polyquaternium-7, polyquaternium-37, polyquaternium-67 and/or polyquaternium-87,
Plant extracts,
Moisture retaining agents,
Perfumes,
UV filters,
Thickening agents such as gelatins or natural gum, such as agar-agar, guar-gum, alginate, xanthan-gum, arabicum gum, karaya gum, locust bean gum, linseed gums, dextranes, cellulose derivatives, e.g. methylcellulose, hydroxyalkylcellulose and carboxymethylcellulose, starch fractions and derivatives, such as amylose, amylopectin and dextrins, clays and layer silicates, e.g. bentonite or fully-synthetic hydrocolloids, such as polyvinylalcohol, Ca—, Mg—Zn soaps,
Structurants such as malic acid and lactic acid,
Dimethylisosorbid,
Cyclodextrine,
Fiber-improving active ingredients, particularly mono-, di- and oligosaccharides, such as glucose, galactose, fructose, fruit sugar and lactose,
Dyes for coloring the agent,
Substances for setting the pH value, for example α- and β-hydroxycarbonic acids, such as citric acid, lactic acid, malic acid, glycolic acid,
Active ingredients such as bisabolol and/or allantoin,
Complexing agents such as EDTA, NTA, β-alanindiessig acid and phosphonic acids,
Ceramides. Ceramides include N-acylsphingosine (fatty acid amides of sphingosin) or synthetic analogs of such lipids (so-called pseudo-ceramides),
antioxidants,
Preservatives, such as sodium benzoate or salicylic acid,
Additional viscosity regulators, such as salts (NaCl).

A second subject matter of the present disclosure is the cosmetic use of the hair cleansing conditions as contemplated herein for the mild cleansing and highly-effective care of hair and scalp, more particularly of brittle, damaged, dull and/or lank hair.

A third subject matter of the present disclosure is the cosmetic use of an active ingredient combination, containing
a) from about 0.01 to about 10 wt. % of at least one polyether-modified polysiloxane,
b) from about 0.01 to about 10 wt. % of at least one amino-functional silicone,
c) from about 0.01 to about 15 wt. % of at least one hydrophobically-modified starch compound and
d) from about 0.10 to about 30 wt. % of at least one amphoteric tenside,
   in hair cleansing conditioners for achieving an optimal balance between a high conditioning effect and a feeling of clean hair.

A fourth subject matter of the present disclosure is the cosmetic use of an active ingredient combination, containing
a) from about 0.01 to about 10 wt. % of at least one polyether-modified polysiloxane,
b) from about 0.01 to about 10 wt. % of at least one amino-functional silicone,
c) from about 0.01 to about 15 wt. % of at least one hydrophobically-modified starch compound and
d) from about 0.10 to about 30 wt. % of at least one amphoteric tenside,
   for improving the skin tolerance of cosmetic (hair) cleansing compositions.

A fifth subject matter of the present disclosure is a cosmetic method for the treatment of hair, wherein the hair cleansing conditioner as contemplated herein is applied to wet hair, massaged in and rinsed off after an exposure time.

The hair cleansing conditioners as contemplated herein apply, mutatis mutandis, to certain embodiments of the use as contemplated herein and of the method as contemplated herein.

The following examples are intended to explain the subject matter of the present disclosure without, however, having any limiting effect.

### Examples

### 1. Composition of the Hair Cleansing Conditioners as Contemplated Herein

The following hair cleansing conditioners as contemplated herein were produced, all numerical values in the examples below—unless otherwise specified—corresponding to the quantity of the respective active ingredient in percentage by weight, relative to the total weight of the agent:

| | Formula 1 | Formula 2 | Formula 3 | Formula 4 | Formula 5 |
|---|---|---|---|---|---|
| Polyether-modified | 0.01-10.00 | 0.025-8.00 | 0.05-6.00 | 0.075-4.00 | 0.10-2.00 |
| polysiloxane a) | | | | | |
| Amino-functional | 0.01-10.00 | 0.025-8.50 | 0.05-7.00 | 0.075-5.00 | 0.10-2.50 |
| silicone | | | | | |
| Hydrophobically- | 0.01-15.00 | 0.10-12.50 | 0.25-10.00 | 0.40-7.50 | 0.50-5.00 |
| modified starch | | | | | |
| compound | | | | | |
| Amphoteric tenside | 0.10-30.00 | 0.50-25.00 | 0.75-20.00 | 1.00-17.50 | 1.50-15.00 |
| Water and other | ad 100 | ad 100 | ad 100 | ad 100 | ad 100 |
| adjuvants and | | | | | |
| additives, where | | | | | |
| applicable | | | | | |
| | Formula 6 | Formula 7 | Formula 8 | Formula 9 | Formula 10 |
| PEG-12 | 0.01-10.00 | 0.025-8.00 | 0.05-6.00 | 0.075-4.00 | 0.10-2.00 |
| Dimethicone and/or | | | | | |
| PEG-14 | | | | | |
| Dimethicone | | | | | |
| Amino-functional | 0.01-10.00 | 0.025-8.50 | 0.05-7.00 | 0.075-5.00 | 0.10-2.50 |
| silicone | | | | | |
| Hydrophobically- | 0.01-15.00 | 0.10-12.50 | 0.25-10.00 | 0.40-7.50 | 0.50-5.00 |
| modified starch | | | | | |
| compound | | | | | |
| Amphoteric tenside | 0.10-30.00 | 0.50-25.00 | 0.75-20.00 | 1.00-17.50 | 1.50-15.00 |
| Water and other | ad 100 | ad 100 | ad 100 | ad 100 | ad 100 |
| adjuvants and | | | | | |
| additives, where | | | | | |
| applicable | | | | | |
| | Formula 11 | Formula 12 | Formula 13 | Formula 14 | Formula 15 |
| Polyether-modified | 0.01-10.00 | 0.025-8.00 | 0.05-6.00 | 0.075-4.00 | 0.10-2.00 |
| polysiloxane a) | | | | | |
| Amodimethicone | 0.01-10.00 | 0.025-8.50 | 0.05-7.00 | 0.075-5.00 | 0.10-2.50 |
| Hydrophobically- | 0.01-15.00 | 0.10-12.50 | 0.25-10.00 | 0.40-7.50 | 0.50-5.00 |
| modified starch | | | | | |
| compound | | | | | |
| Amphoteric tenside | 0.10-30.00 | 0.50-25.00 | 0.75-20.00 | 1.00-17.50 | 1.50-15.00 |
| Water and other | ad 100 | ad 100 | ad 100 | ad 100 | ad 100 |
| adjuvants and | | | | | |
| additives, where | | | | | |
| applicable | | | | | |
| | Formula 16 | Formula 17 | Formula 18 | Formula 19 | Formula 20 |
| Polyether-modified | 0.01-10.00 | 0.025-8.00 | 0.05-6.00 | 0.075-4.00 | 0.10-2.00 |
| polysiloxane a) | | | | | |
| Amino-functional | 0.01-10.00 | 0.025-8.50 | 0.05-7.00 | 0.075-5.00 | 0.10-2.50 |
| silicone | | | | | |
| Hydroxypropyl | 0.01-15.00 | 0.10-12.50 | 0.25-10.00 | 0.40-7.50 | 0.50-5.00 |
| starch phosphate | | | | | |
| Amphoteric tenside | 0.10-30.00 | 0.50-25.00 | 0.75-20.00 | 1.00-17.50 | 1.50-15.00 |
| Water and other | ad 100 | ad 100 | ad 100 | ad 100 | ad 100 |
| adjuvants and | | | | | |
| additives, where | | | | | |
| applicable | | | | | |
| | Formula 21 | Formula 22 | Formula 23 | Formula 24 | Formula 25 |
| Polyether-modified | 0.01-10.00 | 0.025-8.00 | 0.05-6.00 | 0.075-4.00 | 0.10-2.00 |
| polysiloxane a) | | | | | |
| Amino-functional | 0.01-10.00 | 0.025-8.50 | 0.05-7.00 | 0.075-5.00 | 0.10-2.50 |
| silicone | | | | | |
| Hydrophobically- | 0.01-15.00 | 0.10-12.50 | 0.25-10.00 | 0.40-7.50 | 0.50-5.00 |
| modified starch | | | | | |
| compound | | | | | |
| Disodium | 0.10-30.00 | 0.50-25.00 | 0.75-20.00 | 1.00-17.50 | 1.50-15.00 |
| cocoamphodiacetate | | | | | |
| and/or | | | | | |
| cocamidopropyl | | | | | |
| betain | | | | | |
| Water and other | ad 100 | ad 100 | ad 100 | ad 100 | ad 100 |
| adjuvants and | | | | | |
| additives, where | | | | | |
| applicable | | | | | |
| | Formula 26 | Formula 27 | Formula 28 | Formula 28 | Formula 30 |
| PEG-12 | 0.01-10.00 | 0.025-8.00 | 0.05-6.00 | 0.075-4.00 | 0.10-2.00 |
| Dimethicone and/or | | | | | |
| PEG-14 | | | | | |
| Dimethicone | | | | | |
| Amodimethicone | 0.01-10.00 | 0.025-8.50 | 0.05-7.00 | 0.075-5.00 | 0.10-2.50 |
| Hydrophobically- | 0.01-15.00 | 0.10-12.50 | 0.25-10.00 | 0.40-7.50 | 0.50-5.00 |
| modified starch | | | | | |
| compound | | | | | |
| Amphoteric tenside | 0.10-30.00 | 0.50-25.00 | 0.75-20.00 | 1.00-17.50 | 1.50-15.00 |
| Water and other | ad 100 | ad 100 | ad 100 | ad 100 | ad 100 |
| adjuvants and | | | | | |
| additives, where | | | | | |
| applicable | | | | | |
| | Formula 31 | Formula 32 | Formula 33 | Formula 34 | Formula 35 |
| PEG-12 | 0.01-10.00 | 0.025-8.00 | 0.05-6.00 | 0.075-4.00 | 0.10-2.00 |
| Dimethicone and/or | | | | | |
| PEG-14 | | | | | |
| Dimethicone | | | | | |
| Amodimethicone | 0.01-10.00 | 0.025-8.50 | 0.05-7.00 | 0.075-5.00 | 0.10-2.50 |
| Hydroxypropyl | 0.01-15.00 | 0.10-12.50 | 0.25-10.00 | 0.40-7.50 | 0.50-5.00 |
| starch phosphate | | | | | |
| Amphoteric tenside | 0.10-30.00 | 0.50-25.00 | 0.75-20.00 | 1.00-17.50 | 1.50-15.00 |
| Water and other | ad 100 | ad 100 | ad 100 | ad 100 | ad 100 |
| adjuvants and | | | | | |
| additives, where | | | | | |
| applicable | | | | | |
| | Formula 36 | Formula 37 | Formula 38 | Formula 39 | Formula 40 |
| PEG-12 | 0.01-10.00 | 0.025-8.00 | 0.05-6.00 | 0.075-4.00 | 0.10-2.00 |
| Dimethicone and/or | | | | | |
| PEG-14 | | | | | |
| Dimethicone | | | | | |
| Amodimethicone | 0.01-10.00 | 0.025-8.50 | 0.05-7.00 | 0.075-5.00 | 0.10-2.50 |
| Hydroxypropyl | 0.01-15.00 | 0.10-12.50 | 0.25-10.00 | 0.40-7.50 | 0.50-5.00 |
| starch phosphate | | | | | |
| Disodium | 0.10-30.00 | 0.50-25.00 | 0.75-20.00 | 1.00-17.50 | 1.50-15.00 |
| cocoamphodiacetate | | | | | |
| and/or | | | | | |
| cocamidopropyl | | | | | |
| betain | | | | | |
| Water and other | ad 100 | ad 100 | ad 100 | ad 100 | ad 100 |
| adjuvants and | | | | | |
| additives, where | | | | | |
| applicable | | | | | |
| | Formula 41 | Formula 42 | Formula 43 | Formula 44 | Formula 45 |
| Polyether-modified | 0.01-10.00 | 0.025-8.00 | 0.05-6.00 | 0.075-4.00 | 0.10-2.00 |
| polysiloxane a) | | | | | |
| Amino-functional | 0.01-10.00 | 0.025-8.50 | 0.05-7.00 | 0.075-5.00 | 0.10-2.50 |
| silicone | | | | | |
| Hydrophobically- | 0.01-15.00 | 0.10-12.50 | 0.25-10.00 | 0.40-7.50 | 0.50-5.00 |
| modified starch | | | | | |
| compound | | | | | |
| Amphoteric tenside | 0.10-30.00 | 0.50-25.00 | 0.75-20.00 | 1.00-17.50 | 1.50-15.00 |
| Propellant, | 1.00-50.00 | 2.00-40.00 | 3.00-30.00 | 4.00-20.00 | 5.00-17.50 |
| Particularly | | | | | |
| propane/butane | | | | | |
| Water and other | ad 100 | ad 100 | ad 100 | ad 100 | ad 100 |
| adjuvants and | | | | | |
| additives, where | | | | | |
| applicable | | | | | |

| | Formula 46 | Formula 47 | Formula 48 | Formula 49 |
|---|---|---|---|---|
| PEG-12 Dimethicone | 0.50 | 0.40 | 0.50 | 0.40 |
| Amodimethicone | 1.30 | 1.40 | 1.30 | 1.40 |
| Hydroxypropyl starch | 2.50 | 3.50 | 2.50 | 3.50 |
| phosphate | | | | |
| Sodium Cocoamphoacetate | 3.50 | | 3.50 | |
| Cocamidopropyl betain | | 4.00 | | 4.00 |
| Behentrimonium chloride | 3.00 | | 3.00 | |
| Cetrimonium chloride | | 1.00 | | 1.00 |
| Stearyl alcohol | | 6.00 | | 6.00 |
| Cetearyl alcohol | | | | |
| Ceteareth-20 | 0.30 | 0.50 | 0.30 | 0.50 |
| Panthenol | 0.20 | | 0.20 | |
| Citric acid | 1.00 | 1.10 | 1.00 | 1.10 |
| Hydrolyzed keratin | | 0.20 | | 0.20 |
| Perfume | 0.80 | 0.60 | 0.80 | 0.60 |
| Propane-butane | | | 8.00 | 9.00 |
| Preservative | q.s. | q.s. | q.s. | q.s. |
| Water | ad 100 | ad 100 | ad 100 | ad 100 |

### 2. Assessment of the Skin Tolerance of the Hair Cleansing Conditioners as Contemplated Herein

100% healthy women (no allergies or skin problems) between about 18 and about 50 years old (50% from about 18-about 34 years; 50% from about 35-about 50 years) who had tested, in a blind test, the hair cleansing conditioners as contemplated herein against competitor and/or comparison products (in anonymized and coded form) between 29 Oct. and 26 Nov. 2015 where surveyed. Half of the women had used the product as contemplated herein from about 3-about 4 times per week and the other half had used the comparison product.

The skin tolerance of the hair cleansing conditioner as contemplated herein as per Formula 46 was evaluated against a conventional nourishing shampoo (comprising as tenside SLES and cocoamphoacetate and instead hydroxypropyl starch phosphate and silicones a), b) as nourishing agents Polyquaternium-10 and Dimethicone) by employing a dermatological patch test. The results of the patch test can be seen in Tables 1 and 2 below:
a) Treatment with the cleansing conditioner according to Formula 46

TABLE 1

| | | Redness | | | | | | | | Scale | | | | Crack | | | |
|---|---|---|---|---|---|---|---|---|---|---|---|---|---|---|---|---|---|
| Time | No | (erythema) | | | | Edema | | | | formation | | | | formation | | | |
| Intensity | reaction | 1 | 2 | 3 | 4 | 1 | 2 | 3 | 4 | 1 | 2 | 3 | 4 | 1 | 2 | 3 | 4 |
| 6 hours | 18/20 | 2 | 0 | 0 | 0 | 0 | 0 | 0 | 0 | 0 | 0 | 0 | 0 | 0 | 0 | 0 | 0 |
| 24 hours | 19/20 | 0 | 0 | 0 | 0 | 0 | 0 | 0 | 0 | 1 | 0 | 0 | 0 | 0 | 0 | 0 | 0 |
| 48 hours | 19/20 | 0 | 0 | 0 | 0 | 0 | 0 | 0 | 0 | 1 | 0 | 0 | 0 | 0 | 0 | 0 | 0 |
| 72 hours | 19/20 | 0 | 0 | 0 | 0 | 0 | 0 | 0 | 0 | 1 | 0 | 0 | 0 | 0 | 0 | 0 | 0 |

b) Treatment with the aforementioned comparison nourishing shampoo

TABLE 2

| | | Redness | | | | | | | | Scale | | | | Crack | | | |
|---|---|---|---|---|---|---|---|---|---|---|---|---|---|---|---|---|---|
| Time | No | (erythema) | | | | Edema | | | | formation | | | | formation | | | |
| Intensity | reaction | 1 | 2 | 3 | 4 | 1 | 2 | 3 | 4 | 1 | 2 | 3 | 4 | 1 | 2 | 3 | 4 |
| 6 hours | 12/20 | 8 | 0 | 0 | 0 | 0 | 0 | 0 | 0 | 0 | 0 | 0 | 0 | 0 | 0 | 0 | 0 |
| 24 hours | 4/20 | 10 | 5 | 1 | 0 | 1 | 0 | 0 | 0 | 0 | 0 | 0 | 0 | 0 | 0 | 0 | 0 |
| 48 hours | 6/20 | 11 | 1 | 1 | 0 | 0 | 0 | 0 | 0 | 8 | 0 | 0 | 0 | 0 | 0 | 0 | 0 |
| 72 hours | 6/20 | 5 | 0 | 0 | 0 | 0 | 0 | 0 | 0 | 9 | 5 | 0 | 0 | 0 | 0 | 0 | 0 |

The values from Tables 1 and 2 show that the hair cleansing conditioners as contemplated herein have a better skin tolerance than a conventional nourishing shampoo based on anionic sulphate tensides.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1-14. (canceled)

15. A hair cleansing conditioner comprising:
a) from about 0.01 to about 10 wt. % of at least one polyether-modified polysiloxane, relative to the weight of the entire composition;
b) from about 0.01 to about 10 wt. % of at least one amino-functional silicone, relative to the weight of the entire composition;
c) from about 0.01 to about 15 wt. % of at least one hydrophobically-modified starch compound, relative to the weight of the entire composition; and
d) from about 0.10 to about 30 wt. % of at least one amphoteric tenside, relative to the weight of the entire composition.

16. The hair cleansing conditioner of claim 15, wherein the hair cleansing conditioner comprises:
a) from about 0.025 to about 8.00 wt. % of the at least one polyether-modified polysiloxane, relative to the weight of the entire composition;
b) from about 0.025 to about 8.50 wt. % of the at least one amino-functional silicone, relative to the weight of the entire composition;
c) from about 0.10 to about 12.50 wt. % of the at least one hydrophobically-modified starch compound, relative to the weight of the entire composition; and
d) from about 0.50 to about 25.00 wt. % of the at least one amphoteric tenside, relative to the weight of the entire composition.

17. The hair cleansing conditioner of claim 15, wherein the hair cleansing conditioner comprises:
a) from about 0.05 to about 6.00 wt. % of the at least one polyether-modified polysiloxane, relative to the weight of the entire composition;
b) from about 0.05 to about 7.00 wt. % of the at least one amino-functional silicone, relative to the weight of the entire composition;
c) from about 0.25 to about 10.00 wt. % of the at least one hydrophobically-modified starch compound, relative to the weight of the entire composition; and
d) from about 0.75 to about 20.00 wt. % of the at least one amphoteric tenside, relative to the weight of the entire composition.

18. The hair cleansing conditioner of claim 15, wherein the hair cleansing conditioner comprises:
a) from about 0.075 to about 4.00 wt. % of the at least one polyether-modified polysiloxane, relative to the weight of the entire composition;
b) from about 0.075 to about 5.00 wt. % of at least one amino-functional silicone, relative to the weight of the entire composition;
c) from about 0.40 to about 7.50 wt. % of the at least one hydrophobically-modified starch compound, relative to the weight of the entire composition; and
d) from about 1.00 to about 17.50 wt. % of the at least one amphoteric tenside, relative to the weight of the entire composition.

19. The hair cleansing conditioner of claim 15, wherein the hair cleansing conditioner comprises:
a) from about 0.10 to about 2.00 wt. % of the at least one polyether-modified polysiloxane, relative to the weight of the entire composition;
b) from about 0.10 to about 2.50 wt. % of the at least one amino-functional silicone, relative to the weight of the entire composition;
c) from about 0.50 to about 5.00 wt. % of the at least one hydrophobically-modified starch compound, relative to the weight of the entire composition; and
d) from about 1.50 to about 15.00 wt. % of the at least one amphoteric tenside, relative to the weight of the entire composition

20. The hair cleansing conditioner of claim 15, wherein:
a) the at least one polyether-modified polysiloxane is PEG-12 dimethicone and/or PEG-14 dimethicone;
b) the at least one amino-functional silicone is amodimethicone;
c) the at least one hydrophobically-modified starch compound is hydroxypropyl starch phosphate; and
d) the at least one amphoteric tenside is sodium cocoampho(di)acetate and/or cocamidopropyl betain.

21. The hair cleansing conditioner of claim 15, further comprising:
e) from about 0.10 to about 10.00 wt. % of at least one cationic tenside, relative to the weight of the entire composition.

22. The hair cleansing conditioner of claim 21, wherein the hair cleansing conditioner comprises:
e) from about 0.25 to about 9.00 wt. % of the at least one cationic tenside.

23. The hair cleansing conditioner of claim 21, wherein the hair cleansing conditioner comprises:
e) from about 0.40 to about 7.50 wt. % of the at least one cationic tenside.

24. The hair cleansing conditioner of claim 21, wherein the hair cleansing conditioner comprises:
e) from about 0.50 to about 5.00 wt. % of the at least one cationic tenside.

25. The hair cleansing conditioner of claim 21, wherein:
e) the at least one cationic tenside is cetriumonium chloride and/or behentrimonium chloride.

26. The hair cleansing conditioner of claim 15, further comprising:
f) from about 0.10 to about 10.00 wt. % of at least one saturated or unsaturated, linear or branched C₁₂-C₂₄ alcohol, relative to the weight of the entire composition.

27. The hair cleansing conditioner of claim 26, wherein the hair cleansing conditioner comprises:
f) from about 0.50 to about 8.00 wt. % of the at least one saturated or unsaturated, linear or branched C₁₂-C₂₄ alcohol, relative to the weight of the entire composition.

28. The hair cleansing conditioner of claim 26, wherein the hair cleansing conditioner comprises:
f) from about 0.75 to about 7.50 wt. % of the at least one saturated or unsaturated, linear or branched C₁₂-C₂₄ alcohol, relative to the weight of the entire composition.

29. The hair cleansing conditioner of claim 26, wherein:
f) the at least one saturated or unsaturated, linear or branched C₁₂-C₂₄ alcohol is cetyl alcohol, stearyl alcohol and/or cetearyl alcohol.

30. The hair cleansing conditioner of claim 15, wherein the hair cleansing conditioner is free of anionic tensides.

31. The hair cleansing conditioner of claim 15, wherein the hair cleansing conditioner is substantially free of anionic sulphate tensides.

32. A cosmetic use of the hair cleansing conditioner of claim 15 for the mild cleansing and highly-effective care of hair and scalp, more particularly of brittle, damaged, dull and/or lank hair.

33. A cosmetic method for treating hair, wherein the hair cleansing conditioner of claim 1 is applied to wet hair, massaged into the wet hair, and rinsed off after an exposure time.

34. A cosmetic use of an active ingredient combination, comprising:
a) from about 0.01 to about 10 wt. % of at least one polyether-modified polysiloxane;
b) from about 0.01 to about 10 wt. % of at least one amino-functional silicone;
c) from about 0.01 to about 15 wt. % of at least one hydrophobically-modified starch compound; and
d) from about 0.10 to about 30 wt. % of at least one amphoteric tenside;
in hair cleansing conditioners for achieving an optimal balance between a high conditioning effect and a feeling of clean hair or for improving the skin tolerance of cosmetic hair cleansing compositions.
